(19) United States
(12) Patent Application Publication
(10) Pub. No.: US 2025/0223269 A1
(43) Pub. Date: Jul. 10, 2025

(54) SYNTHESIS OF 3,4-BIS(4-AMINO-1,2,5-OXADIAZOL-3-YL)-1,2,5-OXADIAZOLE-N-OXIDE USING 4-AMINO-3-BROMOCARBOHYDROXYMOYL-1,2,5-OXADIAZOLE

(71) Applicants: BRAJA SUNDAR PRADHAN, Hyderabad (IN); NEHA MEHTA, Picatinny Arsenal, NJ (US); KEYUR PATEL, Picatinny Arsenal, NJ (US)

(72) Inventors: BRAJA SUNDAR PRADHAN, Hyderabad (IN); NEHA MEHTA, Picatinny Arsenal, NJ (US); KEYUR PATEL, Picatinny Arsenal, NJ (US)

(21) Appl. No.: 18/407,433

(22) Filed: Jan. 8, 2024

(51) Int. Cl. C07D 271/08

(52) U.S. Cl. CPC C07D 271/08

(57) ABSTRACT

Embodiments of this disclosure describe the synthesis of 4-amino-3 bromocarbohydroxymoyl-1,2,5-oxadiazole from a precursor solution in methanol. The synthesis uses 4-amino-3-aminocarbohydroxymoyl-1,2,5-oxadiazole as the starting material. The procedure involves the sequential treatment of the starting material in methanol with hydrobromic acid, cuprous bromide, and sodium nitrite. This treatment results in the precipitation of a new chemical entity, the bromo-oxime 4-amino-3-bromocarbohydroxymoyl-1,2,5-oxadiazole. Upon obtaining the bromo-oxime derivative, the method further facilitates the production of 3,4-bis(4-amino-1,2,5-oxadiazol-3-yl)-1,2,5-oxadiazole-N-oxide (furoxan derivative) through dimerization in a biphasic reaction medium consisting of ethyl acetate and water, with potassium carbonate serving as a key reactant. This innovative method offers a streamlined approach, enabling the direct use of the intermediate oxadiazole derivative without the need for additional purification, leading to the desired furoxan derivative. This synthesis method stands out for its efficiency, high-purity yield, and fewer steps, offering potential applications in various chemical industries.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter generally relates to the field of furoxan chemistry. More particularly, the present invention relates to a novel, practical method of synthesizing a bromo-oxime derivative to serve as the precursor of choice for the preparation of furoxan derivatives, in particular, 3,4-bis(4-amino-1,2,5-oxadiazol-3-yl)-1,2,5-oxadiazole-N-oxide. The furoxan derivative is used as the penultimate precursor for the preparation of a Hamiltonian material, chemically known as 3,4-bis(4-nitro-1,2,5-oxadiazol-3-yl)-1,2,5-oxadiazole-N-oxide.

### BACKGROUND

The synthesis of furoxan derivatives is of significant interest in oxadiazole chemistry due to its versatile applications. The conventional methods consistently recommend a chloro-oxime derivative, namely, 4-amino-3-chlorocarbohydroxymoyl-1,2,5-oxadiazole, as the precursor of choice for synthesizing furoxan derivatives. Despite these recommendations, numerous attempts to obtain the chloro-oxime moiety following the reported method have failed in our hands. The critical drawback lies in getting the moiety in the acceptable purity for the synthesis. Impurities in the precursor can negatively impact the final product's overall yield, efficiency, and quality, rendering it unsuitable for practical applications. The challenge of obtaining the chloro-oxime moiety in acceptable purity raises concerns about the reliability and reproducibility of the existing synthesis method. These limitations hinder the furoxan derivative's scalability and commercial viability, ultimately limiting its broader utilization in various fields.

The difficulties encountered in preparing the chloro-oxime moiety necessitate the development of an alternative chemical entity that can be predictably synthesized in acceptable purity. An alternative synthesis process that overcomes the limitations of the existing methods is crucial to enable the efficient and cost-effective production of the oxadiazole derivative.

In light of the drawback associated with the existing methods and the pressing need for an alternative approach, the present study aims to introduce a novel, practical, and efficient process for preparing the furoxan precursor using a new chemical entity. The new method promises to overcome the limitations of the chloro-oxime moiety, providing a reliable and streamlined route to obtain high-purity the furoxan derivative, thus contributing significantly to the advancement of oxadiazole chemistry and its diverse applications.

Given the aforementioned challenges, there is a clear need to develop an alternative, safer process that fulfills these requirements.

### SUMMARY

The following invention presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards a synthesis of 4-amino-3-bromocarbohydroxymoyl-1,2,5-oxadiazole (bromo-oxime derivative).

Exemplary embodiments of the present disclosure are directed towards a synthesis of 3,4-bis(4-amino-1,2,5-oxadiazol-3-yl)-1,2,5-oxadiazole-N-oxide (furoxan derivative) using the bromo-oxime derivative.

The present disclosure aims to introduce a new, practical, and efficient method for synthesizing furoxan derivatives.

The objective of the present disclosure is to provide a novel and practical method for the preparation of a furoxan derivative, in particular, 3,4-bis(4-amino-1,2,5-oxadiazol-3-yl)-1,2,5-oxadiazole-N-oxide using the bromo-oxime derivative.

Another objective of the present disclosure is to prepare a furoxan derivative using a new chemical entity named 4-amino-3-bromocarbohydroxymoyl-1,2,5-oxadiazole.

Another objective of the present disclosure is to address the drawbacks associated with the conventional use of the chloro oxime moiety as the precursor of choice, including the inability to achieve the desired purity levels and the subsequent negative impact on the overall yield and quality of the final product.

Another objective of the present disclosure is to significantly improve the purity levels of the precursor used to synthesize furoxan derivatives.

In an exemplary embodiment of the present disclosure, developing a method that utilizes 4-amino-3-bromocarbohydroxymoyl-1,2,5-oxadiazole (bromo-oxime derivative) as the precursor for the synthesis of furoxan derivatives, in particular, 3,4-bis(4-amino-1,2,5-oxadiazol-3-yl)-1,2,5-oxadiazole-N-oxide.

Another exemplary embodiment of the present disclosure may involve developing a method that utilizes furoxan derivatives, in particular, 3,4-bis(4-amino-1,2,5-oxadiazol-3-yl)-1,2,5-oxadiazole-N-oxide as a precursor for the synthesis of 3,4-bis(4-nitro-1,2,5-oxadiazol-3-yl)-1,2,5-oxadiazole-N-oxide.

Another exemplary embodiment of the present disclosure involves evaluating modifications to the Sandmeyer reaction conditions to improve the reaction efficiency and selectivity potentially.

Another exemplary embodiment of the present disclosure may involve precipitating the precursor compound directly from the reaction mixture, minimizing the need for additional purification steps.

Another exemplary embodiment of the present disclosure involves the addition of an aqueous acid drop-by-drop to a stirred solution of 4-amino-3-aminocarbohydroxymoyl-1,2,5-oxadiazole in methanol, resulting in the preparation of 4-amino-3-bromocarbohydroxymoyl-1,2,5-oxadiazole.

Another exemplary embodiment of the present disclosure involves the addition of a copper (I) halide, preferably, copper (I) bromide to the solution to catalyze the formation of the product.

Another exemplary embodiment of the present disclosure may involve dimerization of the 4-amino-3-bromocarbohydroxymoyl-1,2,5-oxadiazole for the preparation of furoxan derivatives, in particular, 3,4-bis(4-amino-1,2,5-oxadiazol-3-yl)-1,2,5-oxadiazole-N-oxide.

Another exemplary embodiment of the present disclosure involves the addition of an aqueous solution of potassium carbonate drop by drop to a stirred solution of 4-amino-3-bromocarbohydroxymoyl-1,2,5-oxadiazole in ethyl acetate, resulting in the preparation of 3,4-bis(4-amino-1,2,5-oxadiazol-3-yl)-1,2,5-oxadiazole-N-oxide.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and so forth, herein do not denote any order, quantity, or importance, but rather are used to distinguish one element from another.

In accordance with the non-limiting exemplary embodiment of the present disclosure, the method of preparing 3,4-bis(4-amino-1,2,5-oxadiazol-3-yl)-1,2,5-oxadiazole-N-oxide (furoxan derivative) using the bromo-oxime derivative may involve a series of interconnected steps.

In the first step, 4-amino-3-aminocarbohydroxymoyl-1,2,5-oxadiazole may be subjected to Sandmeyer reaction conditions. Within these conditions, the compound might be dissolved in methanol to form a clear solution.

Subsequently, this methanolic solution of the compound may then be sequentially treated with an array of reagents. An aqueous solution of hydrobromic acid might be the first to be added, followed by cuprous bromide, and lastly, an aqueous solution of sodium nitrite. When executed under the right conditions, these additions may lead to the successful reaction progression.

Following this series of treatments, a solid might precipitate from the homogenous mixture, indicating a possible successful formation of the desired bromo-oxime derivative.

The resultant solid is then potentially isolated from the mixture through filtration. After filtration, it might be washed rigorously to ensure the removal of residual impurities. Thereafter, drying may yield the bromo-oxime derivative in a relatively pure form. It should be noted that this derivative, once obtained, may not necessarily undergo additional purification steps. Instead, it might be directly utilized in a subsequent step aiming at the potential synthesis of the furoxan derivative.

The culmination of this method may involve the dimerization of the bromo-oxime derivative. This step might be conducted in a biphasic reaction medium, potentially consisting of ethyl acetate and water. In this medium, the derivative may be dissolved in ethyl acetate and treated with an aqueous potassium carbonate solution.

Under the stipulated conditions, this interaction between the bromo-oxime derivative and potassium carbonate may result in a successful dimerization, possibly producing the furoxan derivative with a high degree of purity.

### Working Example: 1

### Preparation of 4-amino-3-bromocarbohydroxymoyl-1,2,5-oxadiazole

In another possible embodiment, aqueous hydrobromic acid (1.34 liter; 48%, w/w) was cautiously added drop by drop to a stirred solution of 4-amino-3-aminocarbohydroxymoyl-1,2,5-oxadiazole (200 g) in methanol (1.5 liter) at room temperature. To the homogenous mixture obtained following the addition, copper (I) bromide (207 g) was added in divided batches at this temperature. The reaction mixture was stirred for 1 hour 15 minutes at room temperature and then cooled to 0° C. An aqueous solution of sodium nitrite (220 g) was added drop by drop at this temperature over 3 hours. After the completion of the addition, the mixture was stirred overnight at this temperature when the reaction was complete, as indicated by thin layer chromatography (tlc). The precipitated solid was filtered, washed with cold water (3×500 ml), and air dried to give the desired product as a solid; yield: 128 g. DSC 214-218° C.; ¹H NMR (DMSO-d₆) 6.2 (br, 2H), 13.59 (s, 1H); ¹³C NMR (DMSO-d₆): 117.3, 142.6, 153.5.

### Working Example: 2

### Preparation of 3,4-bis(4-amino-1,2,5-oxadiazol-3-yl)-1,2,5-oxadiazole-N-oxide

In another potential scenario, an aqueous solution of potassium carbonate (29.45 g) was added drop by drop to a stirred solution of 4-amino-3-bromocarbohydroxymoyl-1,2,5-oxadiazole (50 g) in ethyl acetate (500 ml) at 0° C. On completion of the addition, the mixture was stirred at this temperature for 3 h when the reaction was complete, as indicated by thin layer chromatography (tlc). The mixture was filtered over a pad of celite. The aqueous layer of the filtrate was extracted with ethyl acetate twice. The organic extracts were combined, dried over sodium sulfate, and concentrated under reduced pressure to give the product as a solid; yield: 19.5 g; DSC: 159-164° C.; ¹H NMR (DMSO-d₆): 6.58 (s, 2H), 6.64 (s, 2H); 13C (DMSO-d₆): 104.2, 133.3, 136.2, 146.5, 155.1, 156.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A method of synthesis of 4-amino-3-bromocarbohydroxymoyl-1,2,5-oxadiazole (bromo-oxime derivative), the method comprising:
a) providing a solution of 4-amino-3-aminocarbohydroxymoyl-1,2,5-oxadiazole in methanol;
b) sequentially adding an aqueous solution of hydrobromic acid to the solution, ensuring complete dissolution and formation of a homogenous mixture;
c) introducing cuprous bromide in divided portions to the homogenous mixture, while maintaining the reaction mixture at a controlled temperature;
d) subsequently cooling the reaction mixture and cautiously introducing an aqueous solution of sodium nitrite dropwise, ensuring a maintained low temperature throughout;
e) allowing the reaction to proceed until the formation of a solid precipitate is observed, as indicated by visual inspection or analytic methods;
f) isolating the precipitated solid by filtering, followed by a thorough washing process to ensure the removal of impurities; and
g) drying the obtained solid to yield the desired product, 4-amino-3-bromocarbohydroxymoyl-1,2,5-oxadiazole, in its pure form, ready for subsequent chemical transformations or applications.

2. The method of claim 1, further comprising the synthesis of 3,4-bis(4-amino-1,2,5-oxadiazol-3-yl)-1,2,5-oxadiazole-N-oxide (furoxan derivative) using the 4-amino-3-bromocarbohydroxymoyl-1,2,5-oxadiazole, wherein:
a) a biphasic reaction medium is prepared using ethyl acetate and water;
b) a solution of the 4-amino-3-bromocarbohydroxymoyl-1,2,5-oxadiazole is dissolved in ethyl acetate and introduced into the biphasic reaction medium;
c) an aqueous solution of potassium carbonate is added dropwise to the biphasic reaction medium containing the 4-amino-3-bromocarbohydroxymoyl-1,2,5-oxadiazole solution;
d) allowing the reaction to proceed until a clean dimerization is observed, as indicated by analytic methods;
e) isolating the resultant 3,4-bis(4-amino-1,2,5-oxadiazol-3-yl)-1,2,5-oxadiazole-N-oxide from the biphasic reaction medium by filtration or other suitable separation methods.

3. The method of claim 1, wherein the 4-amino-3-aminocarbohydroxymoyl-1,2,5-oxadiazole is dissolved in methanol at room temperature.

4. The method of claim 1, wherein the sequential treatment with an aqueous solution of hydrobromic acid, cuprous bromide, and sodium nitrite is conducted at a temperature ranging from 0° C. to 25° C.

5. The method of claim 1, wherein the solid is washed using cold water.

6. The method of claim 1, wherein the filtering, washing, and drying steps are performed under an inert atmosphere.

7. The method of claim 1, wherein the solution of the 4-amino-3-bromocarbohydroxymoyl-1,2,5-oxadiazole is prepared at a concentration of between 0.05 to 0.5 moles per liter of ethyl acetate.

8. The method of claim 2, wherein the biphasic reaction medium is prepared with a volume ratio of ethyl acetate to water ranging between 1:1 to 3:1.

9. The method of claim 2, wherein the aqueous solution of potassium carbonate is introduced over a period ranging from 1 to 3 hours.

10. The method of claim 2, wherein the temperature of the biphasic reaction medium during the dimerization is maintained between −5° C. to 10° C.

11. The method of claim 2, wherein the resultant 3,4-bis(4-amino-1,2,5-oxadiazol-3-yl)-1,2,5-oxadiazole-N-oxide is isolated by a combination of filtration followed by extraction using ethyl acetate, ensuring a purity of above 90%.

12. The method of claim 2, wherein the isolated 3,4-bis(4-amino-1,2,5-oxadiazol-3-yl)-1,2,5-oxadiazole-N-oxide undergoes further purification steps such as crystallization or column chromatography to achieve a purity of 98% or higher.

13. A compound characterized by the following structural formula, said compound being an bromo-oxime derivative: [chemical structure]
